(19) United States
(12) Patent Application Publication
(10) Pub. No.: US 2007/0196274 A1
(43) Pub. Date: Aug. 23, 2007

(54) Immunoconjugates with improved efficacy for the treatment of diseases

(76) Inventor: Le Sun, South Plainfield, NJ (US)

Correspondence Address: NIXON PEABODY, LLP, 401 9TH STREET, NW, SUITE 900, WASHINGTON, DC, 20004-2128, US

(21) Appl. No.: 11/655,263

(22) Filed: Jan. 19, 2007

Related U.S. Application Data
(60) Provisional application No. 60/760,383, filed on Jan. 20, 2006.

(51) Int. Cl. A61K 51/00; A61K 39/395; A61K 39/42; C07K 16/10; C07K 16/12; C07K 16/18

(52) U.S. Cl. 424/1.49; 424/155.1; 424/159.1; 424/178.1; 530/388.3; 530/388.4; 530/388.8; 530/391.1

(57) ABSTRACT

The invention provides therapeutic or diagnostic antibodies with modified N— or C-terminal sequences that are enriched with lysine or tyrosine residues. These lysine or tyfosine residues can be used to couple radioisotopes, cytotoxic agents, or detectable labels. The increased stoichiometric ratios of these agents in the antibody conjugates lead to improved therapeutic efficacy or enhanced detection sensitivity. Non-limiting examples of antibodies suitable for the present invention include anti-CD22, anti-ErbB2, anti-VEGF, anti-EGFR, anti-VEGFR, anti-Her-3, anti-Her-4, anti-CEA, anti-CTLA-4, anti-CD4, anti-CD3, anti-CD20, anti-TNF-a, anti-CD11a, anti-Lewis Y antigen, anti-TrailR, anti-IL2R, anti-CD30, anti-CD146, anti-CD147, anti-alpha V integrin beta, anti-CD19, anti-GD2, anti-3H11, anti-EBV, anti-HIV, anti-HBV, anti-HCV, and other disease-specific antibodies.

### CROSS REFERENCE TO RELATED APPLICATIONS

The present application claims priority from and incorporates by reference the entire disclosure of U.S. Provisional Patent Application Ser. No. 60/760,382, filed Jan. 20, 2006.

### TECHNICAL FIELD

The present invention relates to antibodies with modified N— or C-termini that are enriched with lysine or tyrosine residues, and methods of using these antibodies for the treatment or diagnosis of cancer and other diseases.

### BACKGROUND

Antibodies have been used to treat cancer and immunological or angiogenic disorders. The use of antibody-drug conjugates allows targeted delivery of drug moieties to tumors and other diseased tissues, where systemic administration of the unconjugated drug agents may result in unacceptable levels of toxicity to normal cells.

The basic unit of a native antibody is a monomer which consists of two identical heavy chains and two identical light chains linked by disulfide bonds. There are at least five different types of heavy chains—namely, γ, α, δ, μ, and ε, which provide different effector functions. Heavy chains γ, α and δ have three constant domains (C_{H}1, C_{H}2, and C_{H}3), and heavy chains μ and ε have four constant domains (C_{H}1, C_{H}2, C_{H}3, and C_{H}4). Each heavy chain also has one variable domain (V_{H}). There are at least two types of light chain—namely, λ and κ, each of which comprises one constant domain (C_{L}) and one variable domain (V_{L}).

Depending on the amino acid sequences of the constant domains of their heavy chains, native human antibodies can be grouped into five classes: IgG, IgA, IgM, IgD, and IgE. Several of these classes can be further divided into subclasses or isotypes, such as IgG1, IgG2, IgG3, IgG4, IgA, or IgA2. A typical IgG molecule is composed of two heavy chains γ and two identical light chains (λ or κ). Disulfide bonds connect the light chains to the heavy chains, as well as between the heavy chains. The constant domain of the light chain is aligned with the first constant domain of the heavy chain, and the light-chain variable domain (V_{L}) is aligned with the variable domain of the heavy chain (V_{H}) to form the antigen recognition site.

The variability in a variable domain (V_{L} or V_{H}) is not evenly distributed throughout the domain. It is typically concentrated in three segments called hypervariable regions. The more highly conserved portions of a variable domain are called the framework regions (FRs). Each variable domain of native heavy and light chains comprises four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of the antibody. See Kabat et al., SEQUENCES OF PROTEINS OF IMMUNOLOGICAL INTEREST, 5th Ed. Public Health Service, National Institutes of Health, Bethesda, Md. (1991).

Papain digestion of an antibody produces two identical antigen-binding fragments, called Fab fragments, each with a single antigen-binding site, and a residual Fc fragment. Pepsin treatment of an antibody yields an F(ab′)₂ fragment which has two antigen-binding sites joined at the hinge region between C_{H}1 and C_{H}2 through disulfide bonds. The reduction of the F(ab′)₂ fragment produces two Fab′ fragments. Each Fab′ fragment contains at least one sulfhydryl group that can be utilized in conjugation with a toxin, a radioactive isotope, or another agent of interest. Fab′ fragments differ from Fab fragments by the addition of a few residues at the carboxyl terminus of the heavy chain C_{H}1 domain. Fab, Fab′, F(ab′)₂, and other antigen-binding fragments can also be produced using recombinant host cells, as appreciated by those skilled in the art.

Efforts to design and refine antibody-drug conjugates have focused on the selectivity of monoclonal antibodies as well as drug-linking and drug-releasing properties. However, the effectiveness and potency of antibody-drug conjugates are often impaired by the low drug coupling capacity of antibodies. Therefore, there is a need for new methods to improve the coupling capacity of antibodies so as to maximize the therapeutic efficacy of antibody-drug conjugates.

### SUMMARY OF THE INVENTION

The present invention features antibodies with N— or C-termini that are enriched with lysine or tyrosine resides. These lysine or tyrosine residues provide additional moieties for the coupling of cytotoxic agents or detectable labels, leading to improved therapeutic efficacy or detection sensitivity of the antibody conjugates.

In one aspect, an antibody of the present invention comprises an N— or C-terminal sequence which consists of 2 to 20 amino acid residues, a substantial potion of which are lysine or tyrosine residues. The substantial potion can be, for example, at least one-third, one-half, or two-third of the residues in the N— or C-terminal sequence. In one example, the N— or C-terminal sequence consists of 3 to 15, 3 to 10, or 4 to 8 amino acid residues, among which at least 2, 3, 4, 5, 6, or more residues are lysine or tyrosine. These 2, 3, 4, 5, 6, or more residues can be lysine residues. They can also be tyrosine residues. In addition, these 2, 3, 4, 5, 6, or more residues can be a mixture of lysine and tyrosine residues. In another example, the N— or C-terminal sequence consists of about 5, 6, 7, or 8 amino acid residues, among which at least 2, 3, 4, 5, or more residues are lysine or tyrosine.

In many embodiments, the lysine or tyrosine residues in the N— or C-terminal sequence are covalently coupled to radioactive isotopes, chemotherapeutic agents, toxins, prodrugs, pro-drug activating enzymes, or other cytotoxic agents. Non-limiting examples of radioactive isotopes include At²¹¹I¹³¹, I¹²⁵, Y⁹⁰, Re¹⁸⁶, Re¹⁸⁸, Sm¹⁵³, Bi²¹², P³², and Lu¹⁷⁷. Non-limiting examples of cytotoxic agents include mitomycin C and pingyangmycin.

The lysine or tyrosine residues can be enriched at the N— or C-terminus of a light chain of an antibody. These residues can also be enriched at the N— or C-terminus of a heavy chain of an antibody. Where the antibody employed is an antibody fragment (e.g., Fab, F(ab′)₂, or a genetically engineered antibody fragment such as scFv or minibody), the lysine or tyrosine residues can be enriched at the N— or C-terminus of the fragment or a subunit thereof. As used herein, an “antibody” encompasses not only polyclonal and monoclonal antibodies, but also multispecific antibodies (e.g. bispecific antibodies), chimeric antibodies, linear antibodies, reduced antibodies (e.g., rIgG), antibody fragments comprising antigen-binding sites (e.g., Fab fragments, Fab′ fragments, F(ab′)₂, or Fv fragments), single-chain Fv (scFv) molecules, diabodies, triabodies, and minibodies.

In a preferred embodiment, an antibody of the present invention specifically recognizes an antigen selected from the group consisting of a growth factor, a cytokine, a hormone, a growth factor receptor, a hormone receptor, a cytokine receptor, a neurotransmitter receptor, a tyrosine kinase receptor, a tumor-specific or tumor-associated antigen, a G-protein coupled receptor, an ion channel, and an enzyme. Non-limiting examples of antibodies suitable for the present invention include anti-CD22, anti-ErbB2, anti-VEGF, anti-EGFR, anti-VEGFR, anti-Her-3, anti-Her-4, anti-CEA, anti-CTLA-4, anti-CD4, anti-CD3, anti-CD20, anti-TNF-a, anti-CD11a, anti-Lewis Y antigen, anti-TrailR, anti-IL2R, anti-CD30, anti-CD146, anti-CD147, anti-alpha V integrin beta, anti-CD19, anti-GD2, anti-3H11, anti-EBV, anti-HIV, anti-HBV, and anti-HCV. In another preferred embodiment, the antibodies employed in the present invention are humanized or human antibodies.

The present invention also features pharmaceutical compositions which comprise an antibody of the present invention. A typical pharmaceutical composition of the present invention is prepared by mixing an antibody having the desired degree of purity with optional pharmaceutically acceptable carriers, excipients or stabilizers, in the form of lyophilized formulations or aqueous solutions.

In addition, the present invention features methods of using the antibodies of the present invention to modulate cell killing activity or inhibit the growth or activity of cells or pathogens. These methods comprise contacting an antibody of the present invention with the cells/pathogens to be treated. The cells/pathogens to be treated can be cancer cells or other diseased or dysfunctional cells, viruses, bacteria, yeast, fungi, or other disease-causing microbes. The cells can also be healthy cells.

The present invention further features methods of using the antibodies of the present invention to treat cancer or other diseases. These methods comprise administering a therapeutically effective amount of an antibody of the present invention to a subject of interest (e.g., a cancer patient).

Moreover, the present invention features diagnostic kits comprising the antibodies of the present invention. These antibodies are labeled by one or more detectable moieties via the additional lysine or tyrosine residues at the N— or C-termini of the antibodies.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### DETAILED DESCRIPTION OF THE INVENTION

The present invention features therapeutic antibodies with modified C— or N-termini that are enriched with lysine or tyrosine residues. These lysine or tyrosine residues enable the coupling of more radioactive or cytotoxic agents, thereby significantly improving the therapeutic effects of the antibody conjugates. Detectable labels can also be conjugated to the lysine/tyrosine-enriched termini, thereby improving the detection sensitivity of the antibody conjugates.

Antibodies suitable for the present invention include, but are not limited to, monoclonal antibodies, polyclonal antibodies, multispecific antibodies (e.g. bispecific antibodies), chimeric antibodies, linear antibodies, reduced antibodies (e.g., rIgG), Fab fragments, Fab′ fragments, F(ab′)₂, Fv fragments, single-chain Fv (scFv) molecules, diabodies, triabodies, minibodies, and other antibody fragments that comprise antigen-binding sites. The present invention features the use of any antibody isotype, e.g., IgG, IgM, IgA, IgD, or IgE. Preferably, an antibody of the present invention (including Fab, Fv, or other antigen-binding fragments) binds to the target antigen with an affinity of at least 10⁻⁵ M⁻¹, 10⁻⁶ M¹, 10⁻⁷ M¹, 10⁻⁸ M⁻¹, 10⁻⁹ M⁻¹, 10⁻¹⁰ M⁻¹, or stronger. An antibody of the present invention can be a rat, murine, cow, dog, sheep, goat, guinea pig, rabbit, macaque, chimpanzee, chicken, or human antibody. Antibodies derived from other non-human primates, mammals, or vertebrates are also contemplated by the present invention.

Methods for making intact antibodies or antigen-binding fragments are well known in the art. As used herein, “monoclonal” should not be construed as requiring production of the antibody by any particular method. Exemplary methods suitable for making monoclonal antibodies include, but are not limited to, the hybridoma methods (e.g., Kohler et al., NATURE, 256:495 (1975)), the recombinant DNA methods (see, e.g., U.S. Pat. No. 4,816,567), and the phage antibody library methods (e.g., Clackson et al., NATURE, 352:624-628 (1991), and Marks et al., J. MOL. BIOL, 222:581-597 (1991)).

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells used for the production of the monoclonal antibodies serve as a preferred source of such DNA. Once isolated, the DNA may be placed into expression vectors, which are then transfected into host cells such as E. coli cells, simian COS cells, Chinese Hamster Ovary cells, or myeloma cells that do not otherwise produce antibody protein, to obtain the synthesis of monoclonal antibodies in the recombinant host cells. The DNA thus isolated can also be used to prepare chimeric antibodies, antigen-binding fragments, or other antibody derivatives, as appreciated by those skilled in the art.

A chimeric antibody refers to an antibody in which a portion of the heavy or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass. Non-limited examples of chimeric antibodies include those described in U.S. Pat. No. 4,816,567 and Morrison et al., PROC. NATL. ACAD. SCI. USA, 81:6851-6855 (1984).

Preferably, a chimeric antibody of the present invention is a humanized antibody. Humanized antibodies are particularly desirable for therapeutic treatment of human subjects. Humanized forms of non-human (e.g., murine) antibodies are chimeric full-length immunoglobulins, or chimeric antigen-binding fragments (such as Fv, Fab, or F(ab′)₂), which contain minimal sequence derived from non-human immunoglobulin. Humanized antibodies can be derived from human immunoglobulins in which the residues forming the complementary determining regions (CDRs) are replaced by the residues from CDRs of a non-human antibody. In some instances, Fv framework residues of the human immunoglobulin are also replaced by corresponding non-human residues. Humanized antibodies may include residues that are found neither in the recipient antibody nor in the imported CDR or framework sequences. A humanized antibody can comprise at least one or two variable domains, in which all or substantially all of the CDR regions correspond to those of a non-human immunoglobulin and all or substantially all of the constant regions are those of a human immunoglobulin consensus sequence. In many embodiments, a humanized antibody of the present invention comprises at least a portion of a constant region of a human immunoglobulin (Fc). For further details, see Jones et al., NATURE 321:522-525 (1986); Riechmann et al., NATURE 332:323-329(1988); and Presta, CURR. OP. STRUCT. BIOL. 2:593-596 (1992).

The present invention also features the use of human antibodies. Human antibodies can be produced using transgenic mice, which are incapable of expressing endogenous immunoglobulin heavy and light chains but can express human heavy and light chains. The transgenic mice are immunized in the normal fashion with a selected antigen. Monoclonal antibodies directed against the antigen can be obtained using conventional hybridoma technology. The human immunoglobulin transgenes harbored in the transgenic mice rearrange during B cell differentiation, and subsequently undergo class switching and somatic mutation. Using this technique, therapeutically useful IgG, IgA, IgE, or other antibody isotypes can be prepared. Alternatively, phage display technology (McCafferty et al., NATURE 348:552-553 (1990)) can be used to produce intact human antibodies and antibody fragments in vitro, from immunoglobulin variable (V) domain gene repertoires from unimmunized donors. Phage display can be performed in a variety of formats. See, e.g., Johnson, Kevin S. and Chiswell, David J., CURRENT OPINION IN STRUCTURAL BIOLOGY, 3:564-571(1993). Several sources of V-gene segments can be used for phage display. A repertoire of V genes from unimmunized human donors can be constructed and antibodies to a diverse array of antigens (including self-antigens) can be isolated essentially following the techniques described by Marks et al., J. MOL. BIOL., 222:581-597 (1991), or Griffith et al., EMBO J. 12:725-734 (1993). In addition, human antibodies can be generated by in vitro activated B cells (see U.S. Pat. Nos. 5,567,610 and 5,229,275).

Multispecific (e.g., bispecific) antibodies are antibodies that have binding specificities for at least two different epitopes. Traditional production of full length bispecific antibodies is based on the coexpression of two immunoglobulin heavy chain-light chain pairs, where the two chains have different specificities. Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome. According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. In one embodiment, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure can facilitate the separation of the desired bispecific compound from unwanted immunoglobulin chain combinations, as the presence of an immunoglobulin light chain in only one half of the bispecific molecule provides for a facile way of separation.

Multispecific (e.g;, bispecific) antibodies also encompass cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in U.S. Pat. No. 4,676,980, along with a number of cross-linking techniques. Multispecific antibodies can also be produced from recombinant cell culture. See, e.g., Kostelny et al., J. IMMUNOL., 148(5):1547-1553 (1992).

Reduced antibodies are a reduced form of immunoglobulin composed of one complete light chain and one complete heavy chain connected by disulfide bonds. It is essentially one-half of an intact immunoglobulin molecule and contains a single antigen-binding site. Reduced antibodies can be formed by the selective reduction of disulfide bonds in the hinge region of an antibody.

An Fv fragment contains a complete antigen-binding site which includes a V_{L} domain and a V_{H} domain held together by non-covalent interactions. The present invention also features Fv fragments in which the V_{L} and V_{H} domains are cross linked through glutaraldehyde, intermolecular disulfides, or other linkers.

The variable domains of the heavy and light chains can be fused together to form a single chain variable fragment (scFv), which retains the original specificity of the parent immunoglobulin. Preferably, the V_{L} domain is connected to the V_{H} domain by a flexible peptide linker of 5-30 amino acids in length. More preferably, the V_{L} domain is connected to the V_{H} domain by a flexible peptide linker of 10-20 amino acids in length. Highly preferably, the V_{L} domain is connected to the V_{H} domain by a flexible peptide linker of about 15 amino acids in length. Linkers with less than 5, or more than 30, amino acid residues may also be used, provided that they enables the scFv to form the desired structure for antigen binding. For a review of scFv see Plückthun in THE PHARMACOLOGY OF MONOCLONAL ANTIBODIES, vol. 113, Rosenburg and Moore eds., Springer-Verlag, New York, pp. 269-315 (1994).

Diabodies are small antibody fragments with two antigen-binding sites, where each fragment comprises a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Triabodies can be similarly constructed with three antigen-binding sites.

The basic unit of a preferred minibody comprises a V_{L} and a V_{H} domain. In many cases, the basis unit of a minibody also comprises one or more C_{H} or C_{L} domain. For instance, the basic unit of a minibody can be V_{L}-V_{H}-C_{H1}, V_{L}V_{H}-C_{H2}, or V_{L}-V_{H}-C_{H3}. Each minibody may include 1, 2 or more such units to form 1, 2, or more antigen-binding sites.

The antibodies of the present invention comprise modified C— or N-termini that are enriched with lysine or tyrosine residues. In many embodiments, the antibodies of the present invention have an N— or C-terminal sequence consisting of 2 to 30 amino acid residues among which at least two residues are lysine or tyrosine. For instance, the N— or C-terminal sequence can consist of 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues, and at least one-third, one-half, or two-third of these residues are lysine or tyrosine. In one example, the N— or C-terminal sequence consists of 2-20, 2-15, 2-10, 2-8, 2-6, 3-20, 3-15, 3-10, 3-8, 3-6, 4-20, 4-15, 4-10, 4-8, 4-6, 5-20, 5-15, 5-10, 5-8, 6-20, 6-15, 6-10, or 6-8 amino acid residues, among which at least 2, 3, 4, 5, 6, 7, 8, 9, 10, or more residues are lysine or tyrosine. In another example, the N— or C-terminal sequence consists of 3, 4, 5, 6, 7, or 8 amino acid residues, among which at least 2, 3, 4, 5, 6, 7, or 8 residues are lysine or tyrosine. In still another example, the majority of the residues (e.g., at least 2, 3, 4, 5, 6, 7, 8, 9, or 10 residues) in the N— or C-terminal sequence are lysine. In yet another example, the majority of the residues (e.g., at least 2, 3, 4, 5, 6, 7, 8, 9, or 10 residues) in the N— or C-terminal sequence are tyrosine. In a further example, the majority of the residues in the N— or C-terminal sequence consists of a mixture of lysine and tyrosine residues (e.g., at least 2, 3, 4, 5, 6, 7, 8, 9, or 10 residues).

These lysine/tyrosine-enriched terminal sequences can be added to the light or heavy chains of intact antibodies. These terminal sequences can also be added to the antigen-binding fragments of intact antibodies. Preferably, the lysine/tyrosine-enriched sequence is introduced to a C-terminus of an antibody of the present invention. The lysine/tyrosine-enriched sequence can also be added to an N-terminus of the antibody, provided that the addition of the sequence does not significantly change the antigen-binding property of the antibody.

Methods suitable for adding sequences to the N— or C-termini of antibodies or their fragments are well known in the art. Suitable methods include standard recombinant DNA methods, or the use of transgenic hosts (e.g., rat, mice, cow, dog, sheep, goat, guinea pig, rabbit, macaque, chimpanzee, or other non-human primates) in which one or more genes encoding the light or heavy chain(s) are modified. The lysine/tyrosine-enriched terminal sequences can also be chemically linked to the ends of antibodies with desired antigen-binding specificities. In many embodiments, a peptide linker can be fused between the lysine/tyrosine-enriched sequence and the antibody terminus to increase the accessibility of the terminal lysine or tyrosine residues. Preferably, such a peptide linker has about 3, 4, 5, 6, 7, 8, 9, 10, 11, 12, 13, 14, 15, 16, 17, 18, 19, 20, or more amino acid residues.

In another embodiment, a lysine/tyrosine-enriched sequence of the present invention is added within the sequence of an antibody. For instance, a lysine/tyrosine-enriched sequence can be introduced into the junction region between a variable domain and a constant domain, or between two constant domains. A lysine/tyrosine-enriched sequence can also be introduced within a variable or constant domain, provided that the added sequence does not significantly affect the antigen-binding property or the stability of the antibody.

The lysine/tyrosine-enriched terminal sequences can be conjugated with radioactive isotopes, cytotoxic agents, or prodrugs. Suitable radioactive isotopes include, but are not limited to, At²¹¹, I¹³¹, I¹²⁵, Y⁹⁰, Re¹⁸⁶, Re¹⁸⁸, Sm¹⁵³, Bi²¹², P³², and LU¹⁷⁷. Suitable cytotoxic agents include, but are not limited to, chemotherapeutic agents, toxins, or other substances that can inhibit or prevent the function of cells or causes destruction of cells. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, pingyangmycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Sanofi-aventis); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; hormones (such as steroids); anthracycline; vinca alkaloids; mithramycin; neocarzinostatin; macromycin; trenimon; α-amanitin; ricin; ricin A-chain; ethiduim bromide; tenoposide; colchicine; dihydroxy anthracin dione; actinomycin D; diphteria toxin; abrin; arbrin A chain; modeccin A chain; alpha-sarcin; gelonin; mitogellin; retstrictocin; phenomycin; enomycin; α-sarcin; aspergillin; restirictocin; ribonuclease; diphtheria toxin; pseudomonas exotoxin; curicin; crotin; calicheamicin; sapaonaria officinalis inhibitor; maytansinoids; goserelin; glucocorticoids; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide. Toxins suitable for the present invention include small molecule toxins or enzymatically active toxins of bacterial, fungal, plant or animal origin, including fragments and/or variants thereof. Non-limiting examples of suitable toxins include mitomycin C, pingyangmycin, calicheamicin, maytansine (U.S. Pat. No. 5,208,020), trichothene, and CC 1065.

The lysine/tyrosine-enriched terminal sequences can also be used to conjugate prodrugs. A prodrug is a precursor or derivative form of a pharmaceutically active substance that is less cytotoxic to tumor cells than the parent drug and is capable of being enzymatically activated or converted into the more active parent form. See, e.g., U.S. Pat. No. 4,975,287, which is incorporated herein by reference in its entirety. Non-limiting examples of prodrugs include phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

In addition, the lysine/tyrosine-enriched terminal sequences can be used to conjugate prodrug-activating enzymes which convert prodrugs (e.g. a peptidyl chemotherapeutic agent) to active anti-cancer drugs. Enzymes that are suitable for this purpose include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs, β-lactamase useful for converting drugs derivatized with β-lactams into free drugs; and penicillin amidases, such as penicillin Vamidase or penicillin G amidase, useful for converting drugs derivatized at their amine nitrogens with phenoxyacetyl or phenylacetyl groups, respectively, into free drugs.

The lysine/tyrosine-enriched terminal sequences can be conjugated with one type of therapeutic agents (e.g., radioactive isotopes, chemotherapeutic agents, toxins, or prodrugs). The lysine/tyrosine-enriched terminal sequences can also be conjugated with two or more different types of therapeutic agents (e.g., radioactive isotopes+chemotherapeutic agents, radioactive isotopes+toxin, radioactive isotopes+prodrugs, chemotherapeutic agents/toxins+prodrugs, or radioactive isotopes+chemotherapeutic agents/toxins+prodrugs). In addition, two or more different agents of the same type (e.g., two different radioisotopes) can be coupled to a lysine/tyrosine-enriched terminal sequence.

Conjugation of lysine/tyrosine residues with desired agents can be made using a variety of bifunctional or multifunctional cross-linking agents. Non-limiting examples of suitable cross-linking agents include SPDP (N-succinimidyl-3-(2-pyridyldithiol)propionate), EDC (1-Ethyl-3-[3-dimethylaminopropyl]carbodiimide Hydrochloride), AEDP (3-[(2-Aminoethyl)dithio]propionic acide·HCl), ASBA (4-[p-Azidosalicylamido]butylamine), DCC, BMPS (N-[β-Maleimidopropyloxy]succinimide ester), EMCS ([N-e-Maleimidocaproyloxy]succinimide ester), LC-SMCC (Succinimidyl-4-[N-Maleimidomethyl]cyclohexane-1-carboxy-[6-amidocaproate]), LC-SPDP (Succinimidyl 6-(3-[2-pyridyldithio]-propionamido)hexanoate), MBS (m-Maleimidobenzoyl-N-hydroxysuccinimide ester), SIAB (N-Succinimidyl[4-iodoacetyl]aminobenzoate), SMCC (Succiniridyl 4-[N-maleimidomethyl]cyclohexane-1-carboxylate), SMPH (Succinimidyl-6-[13-maleimidopropionamido]hexanoate), Sulfo-SMCC (Sulfosuccinimidyl 4-[N-maleimidomethyl]cyclohexane-1-carboxylate), Sulfo-SMPB (Sulfosuccinimidyl 4-[p-maleimidophenyl]butyrate), Sulfo-NHS-LC-ASA (Sulfosuccinimidyl[4-azidosalicylamido]-hexanoate), Sulfo-SASD (Sulfosuccinimidyl-2-[p-azidosalicylamido]ethyl-1,3′-dithiopropionate), PMPI (N-[p-Maleimidophenyl]isocyanate), NHS-ASA (N-Hydroxysuccinimidyl-4-azidosalicylic acid), SMPT, Sulfo-LC-SPDP, Sulfo-MBS, Sulfo-SIAB, EDC/Sulfo-NHS, and NHS-PEO,-Maleimide (n=2, 4, 8, or 12). This list is not intended to be exhaustive of the various coupling agents known in the art but, rather, is exemplary of the more common coupling agents that may be used.

In many embodiments, the hetero-bifunctional cross-linkers employed contain two reactive groups: one generally reacting with primary amine group (e.g., N-hydroxy succinimide) and the other generally reacting with a thiol group (e.g., pyridyl disulfide, maleimides, or halogens.). Through the primary amine reactive group, the cross-linker can react with the lysine residue(s) of the antibody and through the thiol reactive group, the cross-linker, already tied up to the antibody, reacts with the cysteine residue (or other free sulfhydryl group) on a desired agent (e.g. a cytotoxic or anti-cellular agent).

Antibodies and the agents to be coupled generally have, or are derivatized to have, functional groups available for cross-linking purposes. This requirement is not considered to be limiting in that a wide variety of groups can be used in this manner. For example, primary or secondary amine groups, hydrazide or hydrazine groups, carboxyl alcohol, phosphate, or alkylating groups may be used for binding or cross-linking.

Lysinyl (or amino-terminal) residues are reactive with succinic or other carboxylic acid anhydrides. Derivatization with these agents has the effect of reversing the charge of the lysinyl residues. Other suitable reagents for derivatizing amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate. Modification of tyrosyl residues can be made by reaction with aromatic diazonium compounds or tetranitromethane. For instance, N-acetylimidizole and tetranitromethane can be used to form O-acetyl tyrosyl species and 3-nitro derivatives, respectively. Tyrosyl residues can also be iodinated using I¹²⁵ or I¹³¹.

The lysine/tyrosine residues employed in the present invention can be replaced by cysteine, histidine, arginine, aspartate, or glutamate residues. Cross-linking agents suitable for the conjugation of these residues to the desired therapeutic or diagnostic agents include, but are not limited to, APG (p-Azidophenyl glyoxal monohydrate), ABH (p-Azidobenzoyl hydrazide), TFCS (N-[e-Trifluoroacetylcaproyloxy]succinimide ester), MPBH 4-(4-N-Maleimidophenyl)butyric acid hydrazide hydrochloride, PDPH (3-(2-Pyridyldithio)propionyl hydrazide), and EMCH ([N-e-Maleimidocaproic acid]hydrazide). Each of these residues can be used to replace each and every lysine or tyrosine residues in the modified N— or C-terminal sequences.

Once conjugated, the antibodies of the present invention can be purified to remove contaminants such as unconjugated cytotoxic agents or antibodies. In many cases, it is important to remove unconjugated cytotoxic agents because of the possibility of increased toxicity. Moreover, unconjugated antibodies may be removed to avoid the possibility of competition for the antigen between conjugated and unconjugated species. Numerous purification techniques can be used to provide conjugates to a sufficient degree of purity to render them clinically useful.

The present invention also features antibodies that are conjugated to a “receptor” (such as streptavidin) for utilization in tumor pretargeting, where the antibody-receptor conjugates are administered to the patient, followed by removal of unbound conjugate from the circulation using a clearing agent and then administration of a “ligand” (e.g., avidin) which is conjugated to a cytotoxic agent (e.g., a radio nuclide).

In addition, the antibodies of the present invention can be formulated as immunoliposomes. Liposomes containing antibodies can be prepared by methods known in the art, such as those described in Epstein et al., PROC. NATL. ACAD. SCI. USA, 82:3688 (1985); Hwang et al., PROC. NATL. ACAD. SCI. USA, 77:4030 (1980); and U.S. Pat. Nos. 4,485,045 and 4,544,545. Liposomes with enhanced circulation time are disclosed in U.S. Pat. No. 5,013,556. In one embodiment, an immunoliposome of the present invention is generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Antibody fragments (e.g., Fab′) can be conjugated to the liposomes as described in Martin et al., J. BIOL. CHEM., 257:286-288 (1982) via a disulfide interchange reaction. A cytotoxic agent is optionally contained within the liposome.

In a preferred embodiment, an antibody of the present invention specifically recognizes an antigen selected from the group consisting of tumor-associated antigens, e.g., CEA (carcinoembryonic antigen), CA19-9 (cancer antigen 19-9), CA125 (cancer antigen 125), PSA (prostate specific antigen), beta-HCG (Human chorionic gonadotropin), AFP (Alpha-fetoprotein), AFP-L3 (a lectin-reactive AFP), or thyroglobulin); cell-surface receptors (e.g., growth factor receptors, hormone receptors, G-protein coupled receptors, neurotransmitter receptors, tyrosine kinase receptors, guanylyl cyclase receptors, or ionotropic receptors); ion channels (e.g., voltage gated channels, ligand-gated channels, stretch-activated channels, cyclic nucleotide-gated channels, G-protein-gated channels, inward-rectifier K channels, or light-gated channels); growth factors/cytokines/hormones (e.g., G-CSF, GM-CSF, NGF, neurotrophins, PDGF, FGF, EGF, TNF, IL, steroid or sterol hormone, lipid hormone, peptide hormone, or amine hormone); and enzymes (e.g., kinases, phosphatases, phospholipases, polymerases, telomerases, topoisomerases, or proteases). Non-limited examples of preferred antigens that can be recognized by the antibodies of the present invention include antigens from the following tumors: B-cell lymphoma (e.g., CD19, CD20, CD22, CD25, or HLA DR); Hodgkin's lymphoma (e.g., CD30); acute myeloid leukaemia (e.g., CD33 or CD45); lymphoid disorders (B-cell and T-cell) (e.g., CD52); epithelial tumors (e.g., carcinoembryonic antigen); epithelial tumors (breast, lung, and colon) (e.g., tumor-associated glycoprotein TAG72, epithelial-cell adhesion molecule, or polymorphic epithelial mucin MUCI); colorectal cancer (e.g., A33); clear-cell renal carcinoma (e.g., G250); neuroectodermal tumors (e.g., gangliosides GD2, GD3, GM2); epithelial tumors (breast, lung, colon, prostate, and ovary) (e.g., Lewis Y antigen); ovarian carcinoma (e.g., cancer antigen 125); epithelial tumors (e.g., cancer antigen 19.9, or fibroblast-activation protein); head and neck, lung, breast, and colorectal cancers (e.g., EGFR); glioblastoma multiforme, breast, and lung cancer (e.g., mutant EGFR with exons 2-7 deleted); breast, ovarian, lung, and prostate carcinoma (e.g., HER2/NEU/ERBB2); melanoma (e.g., GD3); and solid tumors (e.g., VEGF or VEGFR2).

An antibody of the present invention can also be selected to be specific to a pathogen, such as virus, bacterium, yeast, fungus, parasite, or other pathogenic or infectious microbes or organisms. The present invention features antibodies against the antigens of the following non-viral organisms: Streptococcus (e.g., Streptococcus agalactiae, Streptococcus pyogenes, Group C streptococci (beta hemolytic, occasionally alpha or gamma, e.g., Streptococcus anginosus or Streptococcus equismilis), Group D streptococci (alpha or gamma hemolytic, occasionally beta, e.g., Streptococcus bovis), Group E streptococci, Group F streptococci (beta hemolytic, e.g., Streptococcus anginosus), Group G streptococci (beta hemolytic, e.g., Streptococcus anginosus), Groups H and K through V streptococci, Viridans streptococci (e.g., Streptococcus mutans or Streptococcus sangui), Streptococcus faecalis, or Streptococcus pneumoniae); Staphylococcus ( e.g., Staphylococcus epidermidis, Staphylococcus aureus, Staphylococcus saprophyticus, Staphylococcus haemolyticus, or Staphylococcus hominis); Actinobacillus (e.g., Actinobacillus lignieresi, Actinobacillus pleuropneumoniae); Actinomyces (e.g., Actinomyces bovis, Actinomyces israelii or Actinomyces naeslundii); Aerobacter (e.g., Aerobacter aerogenes); Alloiococcus (e.g., Alloiococcus otitidis); Anaplasma (e.g., Anaplasma marginale); Bacillus (e.g., Bacillus anthracis or Bacillus cereus); Bordetella (e.g., Bordetella pertussis or Bordetella parapertussis); Borrelia (e.g., Borrelia anserina, Borrelia recurrentis or Borrelia burgdorferi); Brucella (e.g., Brucella canis or Brucella melintensis); Campylobacter (e.g., Campylobacter jejuni); Chlamydia (e.g., Chlamydia psittaci, Chlamydia pneumoniae, Chlamydia trachomatis); Clostridium (e.g., Clostridium botulinum, Clostridium chauvoei, Clostridium difficile, Clostridium hemolyticium, Clostridium novyi, Clostridium perfringens, Clostridium septicum or Clostridium tetani); Corynebacterium (e.g., Corynebacterium equi, Corynebacterium diphtheriae, Corynebacterium pyogenes or Corynebacterium renale); Coxiella (e.g., Coxiella burneti); Cowdria (e.g., Cowdria ruminantium); Dermatophilus (e.g., Dermatophilus congolensis); Erysipelothrix (e.g., Erysipelothrix insidiosa or Erysipelothrix rhusopathiae); Escherichia (e.g., Escherichia coli); Francisella (e.g., Francisella tularenssis); Fusiformis (e.g., Fusiformis necrophorus); Haemobartonella (e.g., Haemobartonella canis); Haemophilus (e.g., Haemophilus influenza, both typable and nontypable, or Haemophilus parainfluenzae); Helicobacter (e.g., Helicobacter pylori) Klebsiella (e.g., Klebsiella pneumoniae); Legionella (e.g., Legionella pneumophila); Leptospira (e.g., Leptospira interrogans); Listeria (e.g., Listeria monocytogenes); Moraxella (e.g., Moraxella bovis or Moraxella catarrhalis); Mycobacterium (e.g., Mycobacterium bovis, Mycobacterium leprae or Mycobacterium tuberculosis); Mycoplasma (e.g., Mycoplasma hyopneumoniae, Mycoplasma gallisepticum or Mycoplasma pneumoniae); Nanophyetus (e.g., Nanophyetus salmincola); Neisseria (e.g., Neisseria gonorrhoeae or Neisseria meninigitidis); Nocardia (e.g., Nocardia asteroides); Pasteurella (e.g., Pasteurella anatipestifer, Pasteurella haemolytica or Pasteurella multocida); Proteus (e.g., Proteus vulgaris or Proteus mirabilis) Pseudomonas (e.g., Pseudomonas aeruginosa); Rickettsia (e.g., Rickettsia mooseria, Rickettsia prowazekii, Rickettsia rickettsii or Rickettsia tsutsugamushi); Salmonella (e.g., Salmonella typhi or Salmonella typhimurium); Shigella (e.g., Shigella dysenteriae or Shigella boydii); Treponema (e.g., Treponema pallidum); Vibrio (e.g., Vibrio cholerae); or Yersinia (e.g., Yersinia enterocolitica or Yersinia pestis); protozoan species selected from Eimeria, Anaplasma, Giardia, Babesia, Trichomonas, Entamoeba, Balantidium, Plasmodium, Leishmania, Toxoplasma, Trypanosoma, Entamoeba, Trichomonas, Toxoplasmosa, or Pneumocystis; fungal species selected from Blastomyces, Microsporum, Aspergillis, Candida, Coccidiodes, Cryptococcus, Histoplasma or Trichophyton; and parasites such as trypanosomes, tapeworms, roundworms, and helminthes.

The present invention also features antibodies against antigens of the following viruses: Paramyxoviridae (e.g., pneumovirus, morbillivirus, metapneumovirus, respirovirus or rubulavirus); Adenoviridae (e.g., adenovirus); Arenaviridae (e.g., arenavirus such as lymphocytic choriomeningitis virus); Arteriviridae (e.g., porcine respiratory and reproductive syndrome virus or equine arteritis virus); Bunyaviridae (e.g., phlebovirus or hantavirus); Caliciviridae (e.g., Norwalk virus); Coronaviridae (e.g., coronavirus or torovirus); Filoviridae (e.g., Ebola-like viruses); Flaviviridae (e.g., hepacivirus or flavivirus); Herpesviridae (e.g., simplexvirus, varicellovirus, cytomegalovirus, roseolovirus, or lymphocryptovirus); Orthomyxoviridae (e.g., influenza A virus, influenza B virus, influenza C virus, or thogotovirus); Parvoviridae (e.g., parvovirus); Picornaviridae (e.g., enterovirus or hepatovirus); Poxviridae (e.g., orthopoxvirus, avipoxvirus, or leporipoxvirus); Retroviridae (e.g., lentivirus or spumavirus); Reoviridae (e.g., rotavirus); Rhabdoviridae (e.g., lyssavirus, novirhabdovirus, or vesiculovirus); or Togaviridae (e.g., alphavirus or rubivirus). Preferred viral antigens include, but are not limited to, antigens from human immunodeficiency virus (HIV), human respiratory syncytial virus, influenza, herpes simplex virus 1 and 2, measles virus, hepatitis A virus, hepatitis B virus (HBV), hepatitis C virus (HCV), smallpox virus, polio virus, west Nile virus, coronavirus associated with severe acute respiratory syndrome (SARS), rotavirus, papilloma virus, papillomaviruses, Epstein-Barr virus (EBV), human T-cell lymphotropic virus type I, and Kaposi's sarcoma-associated herpesvirus.

In one embodiment, an antibody of the present invention specifically recognizes a surface or envelope antigen on the targeted pathogen, such as hepatitis B virus surface antigen (HBVsAg), HIV gp 120 or gp41, and SARS associated coronavirus envelope protein. Antibodies against other viral surface/envelope antigens, as described in the U.S. provisional application Ser. No. 60/760,383, filed Jan. 20, 2006, and entitled “Immunoconjugates for Treatment of Infectious Diseases,” the entire content of which is incorporated herein by reference, are also contemplated by the present invention.

Specific examples of the antibodies of the present invention include antibodies against the following antigens: ErbB receptors (e.g., EGFR/ErbB1, HER2/ErbB2/p185^{neu}, HER3/ErbB3, or HER4/ErbB4/tyro2); BMPR1B (bone morphogenetic protein receptor-type IB); E16 (LAT1 or SLC7A5); STEAP1 (six transmembrane epithelial antigen of prostate); 0772P (CA125 or MUC16); MPF (MSLN, SMR, or megakaryocyte potentiating factor); Napi3b (NAPI-3B, SLC34A2, solute carrier family 34 (sodium phosphate), member 2, or type II sodium-dependent phosphate transporter 3b); Sema 5b (FLJ10372, KIAA1445, Mm.42015, or SEMA5B); PSCA hlg (2700050C12Rik or C530008016Rik); ETBR (endothelin type B receptor); MSG783 (RNF124 or FLJ20315); STEAP2 (HGNC8639, STMP, or prostate cancer associated gene 1); TrpM4 (BR22450 or transient receptor potential cation channel, subfamily M, member 4); CRIPTO (CRIPTO, TDGF1, or teratocarcinoma-derived growth factor); CD21 (Complement receptor 2, or C3d/Epstein Barr virus receptor); CD79b (CD79B or immunoglobulin-associated beta); FcRH2 (IRTA4 or SH2 domain containing phosphatase anchor protein 1a); NCA (CEACAM6); MDP (DPEP1); IL20Rα (ZCYTOR7); Brevican (BCAN or BEHAB); EphB2R (DRT, ERK, EPHT3, or Tyro5); ASLG659 (B7h); PSCA (prostate stem cell antigen precursor); GEDA; BAFF-R (tumor necrosis factor receptor superfamily, member 13C); CD22; CD79a; CXCR5; beta subunit of MHC class II molecule (Ia antigen); P2X5 (purinergic receptor P2X, ligand-gated ion channel, 5); CD72; CD180; FcRH1 (Fc receptor-like protein 1); IRTA2 (FCRL5 or Fc receptor-like 5); or TENB2 (TMEFF2, or transmembrane protein with EGF-like and two follistatin-like domains 2). Other non-limiting examples of the antibodies of the present invention include anti-VEGF, anti-VEGFR, anti-CEA, anti-CTLA-4, anti-CD4, anti-CD3, anti-CD20, anti-TNF-a, anti-CD11a, anti-Lewis Y antigen, anti-TrailR, anti-IL2R, anti-CD30, anti-CD146, anti-CD147, anti-alpha V integrin beta, anti-CD19, and anti-GD2, anti-EBV, anti-HIV, anti-HBV, and anti-HCV.

The present invention further features pharmaceutical compositions comprising the antibodies of the present invention. A typical pharmaceutical composition of the present invention can be prepared by mixing an antibody of the present invention having the desired degree of purity with optional pharmaceutically acceptable carriers, excipients or stabilizers, in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Supplementary active ingredients also can be incorporated into the pharmaceutical compositions of the present invention.

In many embodiments, a pharmaceutical composition of the invention includes a therapeutically effective amount of an antibody comprising a modified N— or C-terminal sequence enriched with lysine or tyrosine residues. As used herein, a “therapeutically effective amount” refers to an amount of an antibody effective to treat a disease or disorder in a mammal. In the case of cancer, the therapeutically effective amount of the drug may reduce the number of cancer cells; reduce the tumor size; inhibit (e.g., slow to some extent and preferably stop) cancer cell infiltration into peripheral organs; inhibit (e.g., slow to some extent and preferably stop) tumor metastasis; inhibit to some extent tumor growth; or relieve to some extent one or more of the symptoms associated with the cancer. To the extent an antibody of the present invention may prevent growth or kill existing cancer cells, it may be cytostatic or cytotoxic. For cancer therapy, efficacy can, for example, be measured by assessing the time to disease progression (TTP) or determining the response rate (RR). Cancers that can be treated using the antibodies of the present invention include, but are not limited to, carcinoma, lymphoma, blastoma, sarcoma, leukemia, and lymphoid malignancies. Specific examples of cancers include squamous cell cancer (e.g. epithelial squamous cell cancer), lung cancer including small-cell lung cancer, non-small cell lung cancer, adenocarcinoma of the lung, squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

The antibodies of the present invention can be combined with other traditional therapies to treat cancers. Agents or factors suitable for use in a combined therapy include many chemical compounds or treatment methods that induce DNA damage when applied to cells. Such agents and factors include radiation and waves that induce DNA damage such as, γ-irradiation, X-rays, UV-irradiation, microwaves, electronic emissions, and the like. Unconjugated chemotherapeutic agents can also be used in combination with an antibody of the present invention.

The active ingredient(s) in a pharmaceutical composition of the present invention can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A pharmaceutical composition of the present invention may be formulated in a neutral or salt form. Pharmaceutically-acceptable salts include the acid addition salts formed with the free amino groups of the protein or formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, compositions or solutions will be administered in a manner compatible with the dosage formulation and in such amount as is therapeutically or prophylactically effective. The formulations are easily administered in a variety of dosage forms such as injectable solutions, drug release capsules and the like. For parenteral administration in an aqueous solution, for example, the solution can be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see, for example, REMINGTON'S PHARMACEUINCAL SCIENCES (15th Edition), pages 1035-1038 and 1570-1580). Some variation in dosage will necessarily occur depending on the condition of the subject being treated.

The skilled artisan is directed to REMINGTON'S PHARMACEUTICAL SCIENCES (15th Edition), Chapter 33, in particular, pages 624-652, the entire contents of which are incorporated herein by reference. Some variation in dosage will necessarily occur depending on the condition of the subject being treated. The person responsible for administration will determine the appropriate dose for the individual subject.

Administration of a pharmaceutical composition of the present invention can be by way of any common route so long as the target tissue is available via that route. This includes oral, nasal, buccal, rectal, vaginal or topical. Alternatively, administration may be by orthotopic, intradermal, subcutaneous, intramuscular, intraperitoneal, intratumoral, circumferentially, catheterization, or intravenous injection.

A pharmaceutical composition can also be administered to a subject of interest (e.g., a cancer patient) parenterally or intraperitoneally. Solutions of the active compounds as free base or pharmacologically-acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include sterile aqueous solutions or dispersions and sterile powders for the extemporaneous preparation of sterile injectable solutions or dispersions. In most cases, the form is sterile and fluid to the extent that easy syringability exists. It is preferably also stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, or vegetable oils. The proper fluidity can be maintained, for example, by the use of a coating, such as lecithin, by the maintenance of the required particle size in the case of dispersion, or by the use of surfactants. The prevention of the action of microorganisms can be brought about by various anti-bacterial or anti-fungal agents, for example, parabens, chlorobutanol, phenol, sorbic acid, thimerosal or the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating an antibody of the present invention in the required amount in an appropriate solvent with various other ingredients enumerated above, as required, followed by filtered sterilization. Generally, dispersions are prepared by incorporating the various sterilized active ingredients into a sterile vehicle, which contains the basic dispersion medium and the required other ingredients from those enumerated above. In the case of sterile powders for the preparation of sterile injectable solutions, the preferred methods of preparation are vacuum-drying or freeze-drying techniques which yield a powder of the active ingredient plus any additional desired ingredient from a previously sterile-filtered solution thereof.

For oral administration, an antibody of the present invention can be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, an antibody of the present invention may be incorporated into an anti-septic wash containing sodium borate, glycerin and potassium bicarbonate. An antibody of the present invention may also be dispersed in dentifrices, including: gels, pastes, powders, or slurries. An antibody of the present invention may be added in a therapeutically or prophylactically effective amount to a paste dentifrice that may include water, binders, abrasives, flavoring agents, foaming agents, or humectants.

The administration of an antibody of the present invention brings the antibody (including the conjugated radioactive isotope(s) or cytotoxic agent(s)) into close contact with the target tissue or cells. These conjugated agent(s) then exert the desired therapeutic effect upon the target tissue or cells. The cells to be treated can be cancer cells or other disease or dysfunctional cells. The cells to be treated can also be healthy cells. The cells or tissues to be treated can also be infected with pathogens, such as viruses, bacteria, parasites, yeast, fungi, or other disease-causing microbes, germs, or worms. The antibodies employed can specifically recognize an antigen of the target cell or pathogen. Preferably, the antigen is a surface antigen, such as an epitope in a cell surface protein, lipid, or saccharide, or in a viral envelope or surface protein.

The antibodies of the present invention can also be used to kill viruses, bacteria, or other pathogens in vitro, through contacting or binding to these pathogens. In addition, the antibodies of the present invention can be used in vitro to kill or modulate the growth or activity of cells infected with these pathogens.

The present invention also features diagnostic kits comprising the antibodies of the present invention. The lysine/tyrosine-enriched end(s) of these antibodies can be used to conjugate detectable labels. Detectable labels suitable for this purpose include a variety of compositions that are detectable by spectroscopic, photochernical, biochemical, immnunochemical, electrical, optical or chemical means. Non-limiting examples include magnetic beads, fluorescent dyes (e.g., fluorescein isothiocyanate, Texas red, rhodamine, green fluorescent protein, and the like), radiolabels (e.g., H³, I¹²⁵, S³⁵, C¹⁴, or P³²), enzymes (e.g., horse radish peroxidase, alkaline phosphatase and others commonly used in an ELISA), and calorimetric labels such as colloidal gold or colored glass or plastic (e.g. polystyrene, polypropylene, latex, etc.) beads.

Means of detecting such labels are well known in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The detection typically involves contacting an antibody of the present invention with a biological sample or a tissue, followed by the detection of binding of the antibody to the target antigen to determine the level of the antigen in the biological sample or tissue. Suitable biological samples can be prepared from biological tissues or fluids. Such samples include, but are not limited to, tissue from biopsy, sputum, aniotic fluid, blood, and blood cells (e.g., white cells). Biological samples can also include sections of tissues, such as frozen sections taken for histological purposes. A biological sample is typically obtained from a multicellular eukaryote, preferably a mammal such as rat, mice, cow, dog, guinea pig, or rabbit, and most preferably a primate such as macaques, chimpanzees, or humans. In one embodiment, a diagnostic kit of the present invention further comprises other reagents (including buffers or control reagents) for conducting immunoassays.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. An antibody comprising an N— or C-terminal sequence which consists of 2 to 20 amino acid residues, wherein said N— or C-terminal sequence includes at least two residues, each of which is selected from the group consisting of lysine and tyrosine.

2. The antibody of claim 1, wherein said N— or C-terminal sequence consists of 3 to 15 amino acid residues.

3. The antibody of claim 1, wherein said N— or C-terminal sequence consists of 3 to 10 amino acid residues.

4. The antibody of claim 1, wherein said N— or C-terminal sequence consists of 4 to 8 amino acid residues.

5. The antibody of claim 1, wherein said N— or C-terminal sequence consists of 6 amino acid residues.

6. The antibody of claim 1, wherein said at least two residues comprise three or more residues, each of which is selected from the group consisting of lysine and tyrosine.

7. The antibody of claim 1, wherein said at least two residues comprise four or more residues, each of which is selected from the group consisting of lysine and tyrosine.

8. The antibody of claim 1, wherein said at least two residues are lysine residues.

9. A The antibody of claim 1, wherein said at least two residues are tyrosine residues.

10. The antibody of claim 1, wherein each of said at least two residues is covalently linked to a radioactive isotope.

11. The antibody of claim 10, wherein said radioactive isotope is selected from the group consisting of At²¹¹, I¹³¹, I¹²⁵, Y⁹⁰, Re¹⁸⁶, Re¹⁸⁸, Sm¹⁵³, Bi²¹², P³², and Lu¹⁷⁷.

12. The antibody of claim 1, wherein each of said at least two residues is covalently linked to a cytotoxic agent.

13. The antibody of claim 12, wherein said cytotoxic agent is selected from the group consisting of a chemotherapeutic agent, a radioisotope, a toxin, and a prodrug.

14. The antibody of claim 13, wherein said cytotoxic agent is selected from the group consisting of mitomycin C and pingyangmycin.

15. The antibody of claim 1, wherein said antibody specifically recognizes an antigen selected from the group consisting of a growth factor, a cytokine, a hormone, a growth factor receptor, a hormone receptor, a cytokine receptor, a neurotransmitter receptor, a tyrosine kinase receptor, a tumor specific antigen, a G-protein coupled receptor, an ion channel, and an enzyme.

16. The antibody of claim 1, wherein said antibody is selected from the group consisting of anti-CD22, anti-ErbB2, anti-VEGF, anti-EGFR, anti-VEGFR, anti-Her-3, anti-Her-4, anti-CEA, anti-CTLA-4, anti-CD4, anti-CD3, anti-CD20, anti-TNF-a, anti-CD11a, anti-Lewis Y antigen, anti-TrailR, anti-IL2R, anti-CD30, anti-CD146, anti-CD147, anti-alpha V integrin beta, anti-CD19, anti-GD2, anti-3H11, anti-EBV, anti-HIV, anti-HBV, and anti-HCV.

17. The antibody of claim 1, wherein said antibody is selected from the group consisting of IgG, IgM, IgA, IgD, and IgE.

18. The antibody of claim 1, wherein said antibody is selected from the group consisting of full-length antibody, scFv, Fv, Fab, F(ab)₂, Fab′, diabody, triabody, and minibody.

19. The antibody of claim 1, wherein said N— or C-terminal sequence is a C-terminal sequence.

20. The antibody of claim 1, wherein said N— or C-terminal sequence is an N-terminal sequence.

21. The antibody of claim 1, wherein said antibody is a humanized or human antibody.

22. The antibody of claim 1, wherein said antibody is a rat, murine, cow, dog, sheep, goat, guinea pig, rabbit, macaque, chimpanzee, or chimeric antibody.

23. A pharmaceutical composition comprising the antibody of claim 1.

24. A method for modulating cell killing activity, or inhibiting the growth of a cell or a pathogen, comprising contacting said cell or pathogen with the antibody of claim 1.

25. The method of claim 24, wherein said cell is a cancer cell, a virus, or a disease-causing microbe.

26. A method for treating a disease in a subject of interest, comprising administering a therapeutically effective amount of the antibody of claim 1 to said subject of interest.

27. The method of claim 26, wherein said subject of interest is a human or an animal, and said diseases is cancer or an infectious disease.
